(19) United States
(12) Patent Application Publication
(10) Pub. No.: US 2014/0356466 A1
(43) Pub. Date: Dec. 4, 2014

(54) METHOD FOR TREATING PAIN AND INFLAMMATION ASSOCIATED WITH ARTHRITIS USING CHROMIUM-THREE CATION IN COMBINATION WITH PHYLLANTHUS EMBLICA AND SHILAJIT

(71) Applicant: Natreon, Inc., New Brunswick, NJ (US)

(72) Inventors: Ramesh C. Gupta, Hopkinsville, KY (US); Aundrea Fleck, Big Sandy, TN (US); Sanyasi R. Kalidindi, Monroe, NJ (US)

(73) Assignee: Natreon, Inc., New Brunswick, NJ (US)

(21) Appl. No.: 14/293,754

(22) Filed: Jun. 2, 2014

Related U.S. Application Data
(60) Provisional application No. 61/829,877, filed on May 31, 2013.

(51) Int. Cl. A61K 36/185

(52) U.S. Cl. CPC A61K 36/185
USPC 424/769

(57) ABSTRACT

Chromium-three cation in combination with Phyllanthus emblica extract and Shilajit is useful in treating symptoms associated with osteoarthritis including reduction of inflammation and pain in a mammal, particularly a human or an animal. The combination shows significant reduction in overall pain levels and other pain measures in a canine model, and in human studies. Thus the composition is an anti-arthritic formulation that decreases pain and inflammation.

This application claims the benefit of earlier filed U.S. Provisional Application No. 61/829,877, filed on May 31, 2013, which is hereby incorporated by reference herein.

### FIELD OF THE INVENTION

The present invention relates to treatment of symptoms associated with osteoarthritis including reduction of inflammation and pain through use of chromium-three cation (Cr 3+) in combination with Phyllanthus emblica extract and the herbo-mineral Shilajit.

### BACKGROUND

Arthritis is a debilitating condition that affects the lifestyle and daily activities of a significant percentage of the human population in the United States and worldwide. According to U.S. CDC statistics (2009), arthritis prevalence estimates found that arthritis is reported by at least one in five adults in every state. Pain is a common symptom of arthritis. Arthritis-attributable severe joint pain is reported by at least one in seven adults with arthritis in every state.

Osteoarthritis (OA) is the most common form of arthritis, affecting millions of people around the world. Often called wear-and-tear arthritis, osteoarthritis occurs when the protective cartilage on the ends of bones wears down over time. While osteoarthritis can damage any joint in the body, the disorder most commonly affects joints in the fingers, hands, neck, lower back, knees and hips. OA begins in the cartilage and eventually causes the two opposing bones to erode into each other. Initially, the condition starts with minor pain during activities, but as the disease progresses the pain can be continuous and even occur while in a state of rest. The pain can be debilitating and prevent one from doing some activities. OA typically affects the weight-bearing joints, such as the knee, back, spine, and pelvis. Osteoarthritis gradually worsens with time, and no cure exists. However, osteoarthritis treatments can slow the progression of the disease, relieve pain and improve joint function.

Unlike rheumatoid arthritis, OA is most commonly a disease of the elderly. Disease onset is gradual and usually begins after the age of 40. More than 30% of women have some degree of OA by age 65. One in two people in the U.S. will experience some form of OA in their lifetime. OA is much more common in women than men and it accounts for more than 50% of arthritis cases in the U.S. (nearly 27 million of the 46 million adults).

OA cannot be cured, but one can prevent the condition from worsening. Pain medications are widely required by individuals with osteoarthritis. Such medications include analgesics such as acetaminophen and NSAIDs (non-steroidal anti-inflammatory drugs). These medications have side effects, which may be serious in some patients. As a result, patients often rely upon natural products with the hope that they are safer than allopathic medications.

Furthermore, Osteoarthritis of both knee and hip joints are common conditions that are associated with disability. Osteoarthritis is the second most common rheumatologic problem and is most frequent joint disease with prevalence of 22% to 39% in India, for example. In this region, knee osteoarthritis affects 6% of adults over 30 years of the Caucasian population and is the most common cause of loco motor disability in the elderly. NSAIDS are the choice of drugs for Osteoarthritis and gastrointestinal toxicity is present in 50% of NSAIDs users. Due to the frequent use of NSAIDS, 5.4% of the patients develop gastritis and peptic ulcers requiring hospitalization. A safer alternative treatment would therefore be beneficial in such cases.

Domestic animals and pets may suffer from this condition as well. There are two types of arthritis that canines suffer from; osteoarthritis and rheumatoid arthritis. Rheumatoid arthritis is less common in the larger breeds, but affects smaller breed canines every day. Osteoarthritis is most common in the larger breeds, but can affect any canine Osteoarthritis is commonly diagnosed within the human world, but is readily overlooked in the canine species. Currently, there are 78.2 million pet dogs in the United States, with one in every five suffering from arthritis, which makes up about 20-25% of pet dogs today (U.S. Pet Ownership Statistics, The Humane Society, 2011). It is a continued struggle to understand the level of pain a canine suffers, since there is an inability for them to communicate how and where they hurt.

Many natural products possess potent antioxidant, anti-inflammatory and cardio-protective properties and are used by patients with increased risk of cardiovascular morbidity and mortality in order to treat or prevent disease and/or reduce symptoms.

Among them, Shilajit is an herbo-mineral drug, which oozes out from a special type of mountain rocks in the peak summer months. It is found at high altitudes ranging from 1000-5000 meters. The active constituents of Shilajit contain dibenzo-alpha-pyrones and related metabolites, small peptides (constituting non-protein amino acids), some lipids, and carrier molecules (fulvic acids). See, Ghosal, S., et al., “Shilajit Part 1—Chemical constituents,” J. Pharm. Sci. (1976) 65:772-3; Ghosal, S., et al., “Shilajit Part 7—Chemistry of Shilajit, an immunomodulatory ayurvedic rasayana,” Pure Appl. Chem. (IUPAC) (1990) 62:1285-8; Ghosal, S., et al., “The core structure of Shilajit humus,” Soil Biol. Biochem. (1992) 23:673-80; and U.S. Pat. Nos. 6,440,436 and 6,869,612 (and references cited therein); all hereby incorporated by reference herein.

Shilajit (PrimaVie®) finds extensive use in Ayurveda, for diverse clinical conditions. For centuries people living in the isolated villages in Himalaya and adjoining regions have used Shilajit alone, or in combination with, other plant remedies to prevent and combat problems with diabetes (Tiwari, V. P., et al., “An interpretation of Ayurvedica findings on Shilajit,” J. Res. Indigenous Med. (1973) 8:57). Moreover being an antioxidant it will prevent damage to the pancreatic islet cell induced by the cytotoxic oxygen radicals (Bhattacharya S. K., “Shilajit attenuates streptozotocin induced diabetes mellitus and decrease in pancreatic islet superoxide dismutase activity in rats,” Phytother. Res. (1995) 9:41-4; Bhattacharya S. K., “Effects of Shilajit on biogenic free radicals,” Phytother. Res. (1995) 9:56-9; and Ghosal, S., et al., “Interaction of Shilajit with biogenic free radicals,” Indian J. Chem. (1995) 34B:596-602). It has been proposed that the derangement of glucose, fat and protein metabolism during diabetes, results into the development of hyperlipidemia. In one study, Shilajit produced significant beneficial effects in lipid profile in rats (Trivedi N. A., et al., “Effect of Shilajit on blood glucose and lipid profile in alloxan-induced diabetic rats,” Indian J. Pharmacol. (2004) 36(6):373-376).

As stated above, many herbs possess potent antioxidant, anti-inflammatory and cardio-protective properties and are used by patients with increased risk of cardiovascular morbidity and mortality in order to treat or prevent disease and/or reduce symptoms. Among them, Phyllanthus emblica (Capros®), syn. Emblica officinalis Gaertn., the Indian gooseberry (PE, “Amla”) is widely used in Indian medicine for the treatment of various diseases. There are studies which show significant anti-hyperglycaemic and lipid lowering effects of PE in diabetic patients. In in-vitro and animal studies, PE demonstrates potent antioxidant effects against several test systems such as superoxide radical and hydroxyl radical scavenging action, and in systemic augmentation of antioxidant enzymes in animals (Antony, et al., “A pilot clinical study evaluate the effect of Emblica officinalis extract (Amlamax™) on markers of systemic inflammation and dyslipidemia,” Indian J. Clin. Biochemistry (2008) 23(4): 378-381).

Further, chromium compounds and supplements containing chromium salts, such as Crominex®3+, are known to improve glucose metabolism. Chromium 3+ (Cr 3+) helps insulin metabolize fat, turn protein into muscle, and convert sugar into energy. Chromium-activated insulin considerably increases the amount of blood sugar available for energy production. This biological effect becomes important as increased glucose in the blood will increase insulin causing an inflammatory response in the peripheral tissues such as the muscles. Inflammation of these tissues may also cause pain in the joints they surround.

Crominex®3+ contains other ingredients including an extract of Phyllanthus emblica (Capros®), and a proprietary form of the adaptogen Shilajit. Crominex®3+ is the safest chromium complex on the market today because it cannot be converted to the toxic compound Cr 6+ (chromium-6 cation, or salts thereof), even in an oxidative environment. No studies have been reported so far regarding the activity of Crominex®3+ in osteoarthritis patients.

In view of the above, it would be desirable to provide a method of using chromium-three cation in combination with Phyllanthus emblica extract and Shilajit for treatment and/or prevention of symptoms associated with osteoarthritis including reduction of inflammation and/or pain in a human patient or in an animal.

### SUMMARY OF THE INVENTION

An objective of the present invention is to develop a method of using chromium-three cation in combination with Phyllanthus emblica extract and Shilajit for treatment and/or prevention of symptoms associated with osteoarthritis including reduction of inflammation and pain in a mammal, particularly a human or an animal.

In one embodiment, a method of treating osteoarthritis in a mammal is provided, comprising administering to the mammal in need thereof a therapeutically effective amount of a chromium-containing composition comprising chromium 3+, an extract of Phyllanthus emblica, Shilajit, and an acceptable carrier. The chromium-containing composition can include from about 400 mcg chromium 3+ to about 1000 mcg chromium 3+ per day. A daily dosage from about 20 mg to about 50 mg per day is equivalent to about 400 mcg to about 1000 mcg of chromium 3+ per day.

In another embodiment, a method of reducing pain and inflammation in an individual afflicted with osteoarthritis is provided, comprising orally administering to the individual in need thereof a therapeutically effective amount of a chromium-containing composition comprising chromium 3+, an extract of Phyllanthus emblica, Shilajit, and an acceptable carrier. The chromium-containing composition can include from about 400 mcg chromium 3+ to about 1000 mcg chromium 3+ per day.

In yet another embodiment, a method of reducing pain and inflammation in a dog is provided, comprising orally administering to the dog in need of such treatment a therapeutically effective amount of a chromium-containing composition comprising chromium 3+, an extract of Phyllanthus emblica, Shilajit, and an acceptable carrier. The chromium-containing composition can include from about 500 mcg chromium 3+ to about 1000 mcg chromium 3+ per day. A daily dosage from about 25 mg to about 50 mg per day is equivalent to about 500 mcg to about 1000 mcg of chromium 3+ per day.

### BRIEF DESCRIPTION OF THE DRAWINGS

FIG. 1 illustrates overall pain levels observed during the canine study in an embodiment according to the present invention.

FIG. 2 illustrates pain levels on manipulation of the limbs observed during the canine study in an embodiment according to the present invention.

FIG. 3 illustrates pain levels after physical exertion observed during the canine study in an embodiment according to the present invention.

### DETAILED DESCRIPTION

In one aspect, the present invention reveals the usefulness of chromium-three cation in combination with Phyllanthus emblica extract and Shilajit in treating symptoms associated with osteoarthritis including reduction of inflammation and pain in a human patient or in an animal.

Crominex®3+, which is a complex of chromium with the polyphenolic compounds of Phyllanthus emblica standardized extract and purified Shilajit, surprisingly exhibited improvement in symptoms associated with osteoarthritis including, but not limited to, inflammation, pain, stiffness, and swelling in and around the joints. Crominex®3+, notably contains very small amounts of Phyllanthus emblica and Shilajit (3 mg of each per 200 mcg (in Cr 3+) dose and 6 mg each per 400 mcg (in Cr 3+) dose, respectively). Both Phyllanthus emblica and Shilajit are usually effective in doses of 250 or 500 mg per dose, but in combination with Chromium, they are surprisingly effective in improving symptoms associated with osteoarthritis including, but not limited to, inflammation, pain, stiffness, and swelling in and around the joints. Cr 3+ is >=200 mcg in 10-12 mg of Crominex®3+ (Natreon, Inc., New Brunswick, N.J.). In one embodiment, a daily dosage from about 20 mg to about 50 mg per day is equivalent to about 400 mcg to about 1000 mcg of chromium 3+ per day.

In an embodiment, the chromium-containing composition comprising Crominex®3+ can include an acceptable carrier such as, for example, microcrystalline cellulose.

In a further embodiment, Crominex (Natreon, Inc., New Brunswick, N.J.) used contains Chromium chloride (CrCl₃.6H₂O), Phyllanthus emblica fruit extract, processed Shilajit and microcrystalline cellulose in a proportion of 1:3:3:3 (wt. ratio).

In the human clinical examples below (Examples B to D) it has been shown that use of Crominex®3+ (400 mcg dose) was effective against a number of osteoarthritis outcome measures.

Animal subjects include large domestic mammals, for example, cows or cattle (or other bovine species), horses, pigs, sheep, goats, other livestock, and the like. Animal subjects may also include smaller domestic mammals, such as, but not limited to, dogs, cats, rabbits, and rodents including rats, mice, hamsters, gerbils, guinea pigs, and the like.

Arthritis does not discriminate between certain species and thus there are many parallels between canine arthritis and human arthritis. Although there are several treatments and preventatives available in the human world, these should not be used on any canine. This is a common occurrence in the veterinary world today, so testing new products to ensure their safety on canines is very important. Arthritis affects an estimated 20-25% of canines in the United States. There are currently approximately 78.2 million pet dogs, with 1 in every 5 adult dogs having arthritis. Veterinarians most commonly prescribe non steroidal anti-inflammatory drugs (NSAIDS) for osteoarthritis treatment and management. Although these treatments are successful in treating osteoarthritis, they also cause many severe side effects. Canines may exhibit a decrease in appetite, gastrointestinal ulcers, and vomiting. NSAIDS have also been linked to long-term hepatotoxic effects. This leads to the importance and need for treatments that do not cause these side effects.

Nutraceuticals such as Crominex®3+ have become a safe and effective alternative for treating arthritis in canines as they do not require a prescription, and offer a safety component with little to no side effects. Nutraceuticals provide a simple alternative to treating arthritis in canines. The canines in the present study were given 25 mg of Crominex®3+ twice daily for duration of 150 days. Every 30 days, each canine was evaluated for overall pain, pain during limb manipulation, and pain after physical exertion. The evaluation of overall pain was based on a scale of 1 to 10 observing the canines overall quality of gait, sitting, lying, body posture, and vocalization. The pain levels during limb manipulation were based on a scale of 0 to 4 observing vocalization, integrity, flexibility, resistance, and presence of crepitance. Pain levels after physical exertion were based on a scale of 0 to 4 observing vocalization, lameness during exercise, pain in limbs, or difficulty rising from a sitting or lying position. Body weight, heart rate, respiration rate, temperature, and serum chemistry biomarkers were evaluated each month to ensure the safety of each canine throughout the 150 days. Radiographs were compared on Days 0 and 150 to observe if Crominex®3+ would reduce osteophytes that may have developed in cranial and caudal limbs and hip joints.

Initial Study Summary

Crominex®3+ is a useful supplement containing Cr 3+ commercially available from Natreon, Inc. (New Brunswick, N.J., USA). Crominex®3+ may be described broadly as a complex of Chromium with the polyphenols in Phyllanthus emblica, such complex (called Chromium emblicate) being incorporated into the fulvic acid structure of Shilajit to improve bioavailability.

Arthritis is one of the most prevalent chronic health problems in the United States, not only in humans, but with different species as well. The purpose of this study is to evaluate the therapeutic efficacy and safety of Crominex®3+ in moderately arthritic canines. Eleven moderately arthritic canines were administered 25 mg of Crominex®3+ twice daily for a total of 150 days. Nine of the canines participated in the treated group and received Crominex®3+ while the other two canines received a placebo. Each month on days 0, 30, 60, 90, 120, and 150 each canine was observed for overall pain levels, pain upon limb manipulation, and pain after physical exertion. Radiographs were also performed on days 0 and 150 to observe if osteophyte formations could be reduced. A safety evaluation was observed each month with serum chemistries to test liver, kidney, and heart muscle functions. Body weight, heart rate, respiration rate, and temperature were also evaluated for any negative changes throughout the entire study.

Ultimately the nine treated canines exhibited a significant reduction in pain levels overall, during limb manipulation, and after physical exertion with P<0.05. Serum chemistries presented with no side effects, and no significant changes in body weight, heart rate, respiration rate, body weight, or temperature were observed during the entire 150 days. The radiographs were unable to provide conclusive evidence that Crominex®3+ was able to reduce osteophytes in a 150 day period. Crominex®3+ has been proven safe and effective in reducing pain in moderately arthritic dogs, and may provide a safer alternative to NSAIDS or any other arthritic medications on the market today.

Through the understanding of canine osteoarthritis, and the treatments available, it is confirmed there is a need for alternative medicine. In this particular study, it is expected that Crominex®3+ will reduce pain associated with arthritis. Although there are limitations on weight, duration, and assumptions relying on owner compliance, research can be conducted and significant conclusions will be reached through subjective and objective parameters.

The purpose of this study was to evaluate the therapeutic efficacy and safety of Crominex®3+ in moderately arthritic canines. Arthritis is one of the most prevalent chronic health problems in the United States, not only in humans, but with different species as well. The most common form of arthritis is osteoarthritis, or degenerative joint disease, characterized by degradation of cartilage, hypertrophy of bones at the margins, decrease in synovial fluid, resulting in pain and stiffness. There are several causes for osteoarthritis, such as hip dysplasia, ruptured cruciate ligament, luxating patellas, trauma, or immune mediated disorders.

Veterinarians most commonly prescribe non steroidal anti-inflammatory drugs (NSAIDS), for osteoarthritis treatment and management. Although these treatments are successful in treating osteoarthritis, they also cause many side effects. Canines may exhibit decrease in appetite, gastrointestinal ulcers, and vomiting. They have also been linked to long-term liver effects. This leads to the importance and need for treatments that do not cause these side effects.

There are alternative treatments such as glucosamine, chondroitin, and other disease modifying agents, which are examples of nutraceuticals. These treatments are characterized as “all natural,” and cause fewer, if any side effects. This makes them more accepted in the veterinary world, and by owners.

Canine Study Protocol

1. The owners will follow the administration instructions properly giving 1 capsule twice daily.

2. The owners have removed their canines from any supplements or treatments that would interfere with the analysis of the anti-arthritic formula at least 14 days prior and will maintain this status throughout the study.

3. The owners will provide an accurate and honest assessment of their daily observations of pain in their canines.

4. Canines are not suffering from any other serious medical conditions such as hepatic or renal disease.

5. Breed, sex, age, and diet have no effect on the efficacy of the anti-arthritic formula.

### DEFINITIONS

As used herein, “NSAID” (Nonsteroidal anti-inflammatory drugs) means a class of drugs that provide pain relief and anti-fever effects. NSAIDS also provide anti-inflammatory effects in higher doses.

As used herein, “nutraceutical” means a constituent of food that is used for human and animal health benefits in treating and preventing diseases.

As used herein, “anti-arthritic formula” can be exemplified by a nutraceutical used in this study, namely Crominex®3+ containing Indian gooseberry (Phyllanthus emblica), and a proprietary form of the adaptogen Shilajit.

As used herein, “osteoarthritis” means a degenerative joint disease that causes inflammation and results in progressive degradation of cartilage in the joints.

As used herein, “moderate arthritis” means the level of arthritis that results in visible signs of pain, reluctance to exercise, decreased flexibility, integrity, and range of motion in the limbs.

As used herein, “pain” can be exemplified by an uncomfortable feeling associated with arthritic changes. Symptoms may include stiffness, limping, decrease in appetite, and vocalization with motion.

As used herein, “crepitance” means a crackling or grating sound often felt or heard when two bones rub against one another.

As used herein, “cartilage” means the elastic but tough connective tissue located at the end of the joint that reduces friction and shock.

As used herein, “joint” means the location in which two or more bones make contact.

Certain limitations or exclusions apply. The study was limited to 11 canines that were determined to be moderately arthritic (i.e., n=11 in test population). The study did not evaluate effects of the anti-arthritic formula on different breeds of dogs or dogs of different ages. The study was limited to canines weighing 40 to 65 pounds. The study was limited to 150 days.

Joints are the main areas where osteoarthritis will occur, so a strong understanding of the joint anatomy will aid in diagnosis and treatment. There are three different categories of joints; fibrous, cartilaginous, and synovial. Fibrous and cartilaginous joints obtain articulation by fibrous tissue or hyaline cartilage allowing little or no movement. Synovial joints obtain articulation united by a synovial joint capsule containing synovial fluid, and are freely movable (Pasquini, C., Spurgeon, T., and Pasquini, S. (2007). Anatomy of domestic animals stemic and regional approach. (11th ed.). Pilot Point: Sudz.). The synovial joint is the main focus in this study as osteoarthritis is most commonly affiliated with the category of synovial joints. The synovial joint capsule contains the viscous synovial fluid, which provides nutrients, lubrication, and reduces friction by creating a cushion for articular cartilage. With osteoarthritis, hyaluronic acid is exhausted and decreases the viscosity of the synovial fluid (Pasquini et al., 2007). Since the cushion has been diminished, friction will increase causing pathological changes in the joints.

There are several types of joints within the skeleton of the canine body, but two are most affected by pathological changes in osteoarthritis. The ball and socket is described by a spherical head fitting into a pit or socket. This allows universal movements such as flexion, extension, abduction, and adduction. The hip and shoulder joints are examples of this type of joint. The second common joint is the hinge joint. This joint is described as a uniaxial joint that allows movement at right angles to the two bones involved. The movements allowed with this type of joint are flexion and extension only. The elbow, carpus, and tarsus joints are examples of this particular type of joint.

Osteoarthritis is best explained as inflammation of the component of a joint causing swelling and pain. A synonymous term is degenerative joint disease, and is the most prevalent of all the types of arthritis debilitating canines today. Osteoarthritis is a progressive deterioration of the articular cartilage in the joints, which may cause joint effusion, and bone spurs called osteophytes around the margins of the joints. This type of arthritis may also occur due to excessive uncontrolled inflammation around the joints from soft tissue swelling. There are two types of osteoarthritis, primary and secondary. Primary osteoarthritis is characterized by normal aging, or wearing of the cartilage in the joint. Secondary osteoarthritis is characterized as a result from an underlying cause such as hip dysplasia. Both types lead to the loss of cartilage in the joint and the cartilage producing cells. These observations apply to mammals including canines and humans.

The extracellular matrix of cartilage is made up of type II collagen and proteoglycans. The body will continually remodel cartilage to maintain a proper volume, but in osteoarthritis the cartilage degrading enzymes matrix metalloproteinases take over. Chondrocytes respond to the loss of cartilage by propagating type II collagen and proteoglycans and the cartilage becomes thick for several years, but degradation will eventually occur.

Exemplary Disorder: Arthritis in Canines

For example, in canine species there are two types of arthritis that canines suffer from; osteoarthritis and rheumatoid arthritis. Rheumatoid arthritis is less common in the larger breeds, but affects smaller breed canines every day. Osteoarthritis is most common in the larger breeds, but can affect any canine. Osteoarthritis is commonly diagnosed within the human world, but is readily overlooked in the canine species. Currently, there are 78.2 million pet dogs in the United States, with one in every five suffering from arthritis, which makes up about 20-25% of pet dogs today. It is a continued struggle to understand the level of pain a canine suffers, since there is an inability for them to communicate how and where they hurt.

Canines experiencing joint pain exhibit signs of lethargy, inappetence, weight gain, reluctance to exercise, lameness, and tenderness on palpation of affected joints. Most commonly they will exhibit reluctance to jump on furniture or have difficulty in rising from a lying or sitting position. As with humans, there are several factors that cause arthritis, or cause it to progress more quickly: 1) canines that are over the age of five years, 2) trauma, 3) malnourishment, 4) obesity, 5) breed, and 6) those with a higher level of activity typically suffer from arthritis early in life. Even though there are several signs canines exhibit, diagnosing the debilitating disease provides confirmation, and allows proper treatment options to be assessed.

The same diagnostic instruments are available in veterinary medicine as in human medicine. Common diagnostic tools include radiographs, magnetic resonance imaging (MRI), synovial fluid evaluation, patient history, and physical examination. Radiographs allow an internal image to visualize physiological changes such as calcifications, synovial fluid loss, bone degradation, and bony growths such as osteophytes. Synovial fluid can be evaluated for viscosity, volume, color, turbidity, and protein content. The patient's signalment or history can aid in diagnosing as certain breeds such as German Shepherds are genetically predisposed to hip and/or elbow dysplasia. The physical examination is the most important and most common tool used to diagnose canine arthritis. Most veterinary clinics may not have the expensive diagnostic equipment and rely heavily on physical examination.

Limb manipulation is readily used to evaluate range of motion, flexibility, and crepitance. Crepitance is the “crunching” feeling in a joint and is a common sign of arthritis. Other tests such as Ortiloni and cranial tibial drawer can be performed to test the integrity of a joint. Ortiloni is performed on the hip joint by flexing the knee and hip to ninety degrees, placing the index finger on the greater trochanters, and abducting the hip (Ortolani's sign. In (2007). Saunders Comprehensive Veterinary Dictionary (3rd ed.). Elsevier, INC.). As the hip is abducted, or moved away from the body, a positive Ortiloni will present with a “clunk” sound or feeling as the femoral head relocates anteriorly to the acetabulum, or hip socket. The cranial tibial drawer can also be performed to diagnose the rupture of the cranial cruciate ligament (CCL). In this procedure, the canine is in lateral recumbency with the veterinarian located behind the patient. The thumb of one hand is placed on the caudal aspect of the femoral condular region, and the index finger of the same hand is placed over the patella. The thumb of the other hand is placed on the head of the fibula and the index finger is placed on the tibial crest (Devine, S. B. (1993). Cranial tibial thrust: a primary force in the canine stifle. J Am Vet Med Assoc., 183(4), 456-459). A positive tibial drawer is elicited with the ability to move the tibia cranially, or forward, in respect to the fixed femur. Although there is a continuous struggle to diagnose canine arthritis, many tests and resources are available. Once a diagnosis is made, the veterinarian can begin the battle for treatment and management of canine arthritis.

There are several options that a veterinarian will use to attempt to combat the chronic debilitating disease of canine arthritis. Medicinal treatments include non steroidal anti-inflammatory drugs (NSAIDS), corticosteroids, nutraceuticals, surgery, and other less common methods. A combination of these treatment plans may be more beneficial in aiding our canine friends through this painful disease.

For example, nutraceuticals are commonly used to reduce pain and inflammation. This is achieved by inhibiting the formation of destructive enzymes and prostaglandins in arthritic joints, and increasing constituents such as synovial fluid.

Nutraceuticals are typically used as food additives or made into supplementation products. They can be food alone such as garlic cloves, or parts of food like omega-3 fatty acids in the oils from fish. Other forms of nutraceuticals are glucosamine HCl, chondroitin sulfate, mehtylsulfonylmethane (MSM), boswellic acid, and specific chemical elements (Curtis, C. L., Harwood, J. L., Dent, C. M., and Caterson, B. (2004). Biological basis for the benefit of nutraceutical supplementation in arthritis. Drug Discovery Today, 9(4), 165-172).

These products have become more popular in veterinary medicine as they attempt to obtain a desirable therapeutic outcome with reduced or no side effects. The anti-arthritic formula in this study Crominex®3+ is classified as a nutraceutical, utilizing chemical elements and other constituents. Crominex®3+ is categorized as a nutraceutical and is used as an analgesic for pain associated with canine arthritis, for example, or human arthritis. Other benefits include normal cellular glucose transport, healthy endothelial function, healthy lipid and triglyceride levels, and normal cellular energy production.

Crominex®3+ contains elemental chromium (Cr 3+) which is known for improving glucose metabolism. As previously stated, this action becomes important as increased glucose in the blood will increase insulin causing an inflammatory response in the peripheral tissues such as the muscles. Inflammation of these tissues may also cause pain in the joints they surround. Other ingredients in Crominex®3+ include an extract from Indian gooseberry tree (Phyllanthus embilica), and a proprietary form of the adaptogen Shilajit.

Canine Study Design

The purpose of this study was to observe and analyze the effects of Crominex®3+ in moderately arthritic canines Analyzing radiographs and other variables, results over a 150 day period can be compared to the side effects and use of other medications such as NSAIDS or corticosteroids. Through collection of subjective data, such as results from limb manipulation and overall gait, and objective data such as vitals and blood serum chemistries, nutraceuticals are shown to be a suitable alternative to other medications yielding fewer to no side effects. The main objectives of this research project were as follows:

1. Determine if Crominex®3+ decreases pain and inflammation caused by arthritis based on subjective data, such as overall pain, pain upon limb manipulation, and pain after physical exertion.

2. Determine if Crominex®3+ may increase flexibility and range of motion in the affected joints.

3. Evaluate radiographs to determine if affected joints exhibited improvements after 150 days.

4. Determine the safety of Crominex®3+ by observing total health with heart rate, respiration rate, body temperature, and liver, kidney, and heart functions.

Population and Sample.

Eleven moderately arthritic canines were selected to participate in the study based on the limitations stated above such as weight and level of arthritis they exhibited. The sources used to obtain participants included the Humane Society of Calloway County, local veterinary clinics, and faculty and students in the Animal Health Technology program at Murray State University. All owners of the canines were required to sign a consent form agreeing to administer the anti-arthritic formula orally as directed, meet with the researcher every 30 days where blood collection, radiographs, physical exam, and observational examinations would occur. In addition, any canines that were on any other arthritic medications were asked to discontinue administration at least 14 days prior the beginning of the study on day zero. All eleven canines participated in the complete duration of the study; hence the population and the sample are equivalent.

The location remained the same throughout the study at the Carman Pavilion at Murray State University (Murray, Ky., USA). This location provided all the proper equipment to conduct the analysis, and provided a neutral location for all participants to transport their canines. The BVC lab provided a large space for physical examination, limb manipulation, and the centrifuge to spin blood samples. The radiography room provided the equipment to perform and view radiographs of affected areas. Weight in Kg was observed with the electronic scale, and the parking lot provided a large space for physical exertion. Research was performed over the weekends when school is not in session.

The particular research design that will best represent the data collected is a control group time series design. After random assignment, nine of the canines were selected to receive the anti-arthritic formula and became the treated group, and two canines were selected to receive the placebo, became the control group. Each canine was observed every 30 days for the entire duration of 150 days. Subjective and objective data with baseline values were collected on day 0. The design of this study is depicted in tabular form below.

The particular research design that will best represent the data collected is a control group time series design. After random assignment, nine of the canines were selected to receive the anti-arthritic formula and became the treated group, and two canines were selected to receive the placebo, became the control group. Each canine was observed every 30 days for the entire duration of 150 days. Subjective and objective data with baseline values were collected on day 0. The design of this study is depicted in TABLE 1 below.

TABLE 1
Simple Time Series Design

| | Date | | | | | | | | | | |
|---|---|---|---|---|---|---|---|---|---|---|---|
| | | | Oct. 27, 2012 | | Dec. 1, 2012 | | Jan. 5, 2013 | | Feb. 2, 2013 | | Mar. 2, 2013 |
| | Sep. 29, 2012 | 30 | Day | 30 | Day | 30 | Day | 30 | Day | 30 | Day |
| | Day 0 | Days | 30 | Days | 60 | Days | 90 | Days | 120 | Days | 150 |
| Treated | Obs | Tx | Obs | Tx | Obs | Tx | Obs | Tx | Obs | Tx | Obs |
| Control | Obs | Tx | Obs | Tx | Obs | Tx | Obs | Tx | Obs | Tx | Obs |

Obs: (Observation)
Tx: (Treated)

All data were collected from each of the eleven canines (dog subjects A-K) every 30 days for the entire duration of 150 days. Subjective and objective data were collected during the complete physical examinations.

Subjective data. There were three main scales used to observe pain in the arthritic canines:

1. Overall pain was observed on a scale of 1 to 10 (1=slight pain, and 10=constant or severe pain). Observing overall quality of gait, rising from a sitting or lying position, and lying from a standing position were used for the evaluation.

2. Pain during limb manipulation was observed on a scale of 0 to 4 and was based on vocalization, body posture, flexibility, and resistance. Moderately arthritic canines would range around 2-2.5 on this scale. Each limb was assessed in lateral recumbency, observing also crepitance, flexibility, and integrity of the joints with flexing and extending motions.

3. Pain after physical exertion was observed on a scale of 0 to 4 and was based on vocalization, body posture, flexibility, and resistance. Moderately arthritic canines would range around 2-2.5 on this scale. Each canine was jogged for a total of two minutes. Throughout exercise, canines were evaluated for any evidence of exercise intolerance or lameness.

Objective data: Objective data were recorded during physical examination every 30 days. Heart and respiration rates were observed with a stethoscope measuring beats per minute. Body temperature was recorded with a rectal digital thermometer, and body weight was measured with an electronic scale.

Blood collection. Blood samples were collected from the jugular vein with a 22 gauge needle and 3 ml syringe. The sample was placed into a 4 ml serum separator tube and allowed to clot for 10 minutes. Each tube was then centrifuged at 10,000 RPM for 10 minutes, and the serum was placed into a properly labeled red top tube. The serum was then frozen until it was transported to the Breathitt Veterinary Center where it was processed. Liver, kidney, and heart evaluations were assessed using serum biomarkers every 30 days to ensure the safety of the anti-arthritic formula.

All data were subjected to Number Cruncher Statistical Systems (NCSS) 2000 for Windows®. Data were analyzed using one-way ANOVA coupled with Tukey-Kramer test. Values with p<0.05 were considered as significantly different compared to pretreated values of Day 0. Graphical data was performed using Microsoft Excel. The advantages to using a statistical program are to allow the computer to compute the statistical math and yield precise, and rapid results. Excel allows data to be entered and will automatically generate a graph to represent the entered data.

Reliability and Validity. As with any research study, precautions need to be taken to ensure credibility and trustworthiness of the data and analyzing the data. In this particular study, there were several things that ensured validity, such as collecting blood from the jugular vein. This also ensured no hemolysis occurred, which would falsely elevate the serum chemistry levels. With subjective data such as limb manipulation, performing the exams the same way each month increased consistency when numerical data were placed on subjective observations. Lastly, radiographs were taken on day 0 and day 150 so it was important to use the same technique to ensure the comparison was accurate when evaluating changes.

Results and Discussion

A total of nine canines were placed into the treatment group and were given the anti-arthritic formula. Each owner was instructed to administer one 25 mg capsule of Crominex®3+ orally once before the morning meal and once before the evening meal for a total of 150 days. A total of two canines were placed in the control group and received the placebo capsule and owners were also instructed to administer the capsules twice daily before meals for 150 days. Owners met with the researcher every 30 days at the Carman Pavilion at Murray State University and observations were made on days 0, 30, 60, 90, 120, and 150 days. Each canine was evaluated for overall pain by observing pain from rising from a lying or sitting position, overall gait, and lowering into a sitting position. Pain upon limb manipulation was also evaluated by observing flexibility, integrity, crepitance, lameness, and pain after physical exertion. Radiographs were taken on days 0 and 150 and were compared to observe any internal changes over the 150 day treatment. Blood samples were collected every 30 days and the serum samples were analyzed for changes in liver, kidney, and cardiac muscle function.

The canines in the treatment group exhibited a significant reduction in overall pain, pain upon limb manipulation and pain after physical exertion on days 90, 120, and 150. Values were considered significant as compared to Day 0 (P<0.05). Body weight, heart rate, respiration rate, temperature, and blood serum chemistry levels were recorded and changes were not considered statistically significant (P>0.05). No side effects were noted in any canine during the entire 150 days.

All data were recorded and analyzed statistically and the mean±standard error of means were calculated for each parameter every 30 days for both the treated and placebo groups. All values are shown in tables 2 through 17. Some values could not be calculated, such as the respiration rates due to panting participants.

Overall Pain.

Overall pain was evaluated in eleven canines over a period of 150 days in increments of 30 days, including baseline values at Day 0. Levels of overall pain were observed on a scale of 1 to 10, with 1=slight pain, 5=moderate pain, and 10=severe and constant pain. Determination of pain levels were observed by quality of overall gait, rising from sitting or lying positions, lying from a standing position, posture, vocalization and observations during physical exertion. Patient history and owner's responses in a monthly questionnaire were considered when determining pain levels.

Results, as shown in Table 2 below, show baseline values of overall pain on Day 0 (6.00±0.50) with moderately arthritic pain in 9 canines Overall pain levels by Day 90 were significantly (P<0.05) decreased (3.11±0.31). Days 120 and 150 presented further reduction in pain levels (2.44±0.29; and 1.55±0.18, respectively).

TABLE 2
Effects of Crominex ®3+ on Overall Pain Level in Arthritic Canines

| Day | Dog | Level | Mean ± SEM |
|---|---|---|---|
| 0 | A | 7 | 6.00 ± 0.50 |
| | B | 4 | |
| | C | 7 | |
| | D | 5 | |
| | E | 4 | |
| | F | 5 | |
| | G | 8 | |
| | H | 7 | |
| | I | 7 | |
| 30 | A | 7 | 5.11 ± 0.48 |
| | B | 4 | |
| | C | 6 | |
| | D | 4 | |
| | E | 3 | |
| | F | 4 | |
| | G | 7 | |
| | H | 5 | |
| | I | 6 | |
| 60 | A | 6 | 4.44 ± 0.38 |
| | B | 4 | |
| | C | 5 | |
| | D | 3 | |
| | E | 3 | |
| | F | 4 | |
| | G | 6 | |
| | H | 4 | |
| | I | 5 | |
| 90 | A | 4 | 3.11 ± 0.31* |
| | B | 3 | |
| | C | 3 | |
| | D | 2 | |
| | E | 2 | |
| | F | 2 | |
| | G | 4 | |
| | H | 4 | |
| | I | 4 | |
| 120 | A | 3 | 2.44 ± 0.29* |
| | B | 2 | |
| | C | 2 | |
| | D | 2 | |
| | E | 1 | |
| | F | 2 | |
| | G | 3 | |
| | H | 3 | |
| | I | 4 | |
| 150 | A | 2 | 1.55 ± 0.18* |
| | B | 1 | |
| | C | 1 | |
| | D | 1 | |
| | E | 1 | |
| | F | 2 | |
| | G | 2 | |
| | H | 2 | |
| | I | 2 | |

*Significantly different from the value of Day 0 (P < 0.05)

Results, as shown in Table 3 below, present the two canines that were treated with the placebo and exhibited no significant decrease in overall pain levels as compared to the baseline values on Day 0.

TABLE 3
Effects of Placebo on Overall Pain Level in Arthritic Canines

| Day | Dog | Level | Mean ± SEM |
|---|---|---|---|
| 0 | J | 5 | 5.5 ± 0.24 |
| | K | 6 | |
| 30 | J | 5 | 5.5 ± 0.24 |
| | K | 6 | |
| 60 | J | 4 | 5.0 ± 0.47 |
| | K | 6 | |
| 90 | J | 4 | 5.0 ± 0.47 |
| | K | 6 | |
| 120 | J | 4 | 5.0 ± 0.47 |
| | K | 6 | |
| 150 | J | 5 | 5.5 ± 0.24 |
| | K | 6 | |

Pain During Limb Manipulation.

Pain during limb manipulation was evaluated in eleven canines over a period of 150 days in increments of 30 days, including baseline values on Day 0. Levels of pain from limb manipulation were observed on a scale of 0 to 4 with moderately arthritic canines exhibiting levels at approximately 2-2.5. Each limb was assessed in lateral recumbency observing vocalization, body posture, flexibility, integrity, and presence of crepitance. The Ortiloni and cranial tibial thrust examination were preformed as well.

As shown in Table 4 below, results for baseline values observed on Day 0 present at an overall mean of (2.50±0.14) concluding the 9 treated canines were observed to be moderately arthritic during limb manipulation. Pain levels by Day 90 were significantly (P<0.05) decreased (1.58±0.17). Pain levels on Days 120 and 150 were further decreased (1.14±0.15; 0.78±0.08, respectively).

TABLE 4
Effects of Crominex ®3+ on Pain from
Limb Manipulation in Arthritic Canines

| Day | Dog | Level | Mean ± SEM |
|---|---|---|---|
| 0 | A | 3 | 2.50 ± 0.14 |
| | B | 2.5 | |
| | C | 3 | |
| | D | 2 | |
| | E | 2 | |
| | F | 2 | |
| | G | 2.5 | |
| | H | 3 | |
| | I | 2.5 | |
| 30 | A | 2.75 | 2.19 ± 0.22 |
| | B | 2 | |
| | C | 3 | |
| | D | 1.5 | |
| | E | 1 | |
| | F | 2 | |
| | G | 2.25 | |
| | H | 2.75 | |
| | I | 2.5 | |
| 60 | A | 2.5 | 1.81 ± 0.20 |
| | B | 2 | |
| | C | 2.5 | |
| | D | 1.5 | |
| | E | 0.75 | |
| | F | 1 | |
| | G | 2 | |
| | H | 2 | |
| | I | 2 | |
| 90 | A | 2 | 1.58 ± 0.17* |
| | B | 1.75 | |
| | C | 2 | |
| | D | 1 | |
| | E | 0.75 | |
| | F | 1 | |
| | G | 2 | |
| | H | 2 | |
| | I | 1.75 | |
| 120 | A | 1.5 | 1.14 ± 0.15* |
| | B | 1 | |
| | C | 1.5 | |
| | D | 0.75 | |
| | E | 0.5 | |
| | F | 0.75 | |
| | G | 1 | |
| | H | 1.75 | |
| | I | 1.5 | |
| 150 | A | 1 | 0.78 ± 0.08* |
| | B | 1 | |
| | C | 0.75 | |
| | D | 0.5 | |
| | E | 0.5 | |
| | F | 0.75 | |
| | G | 0.5 | |
| | H | 1 | |
| | I | 1 | |

*Significantly different from the value of Day 0 (P < 0.05)

Results, as shown in Table 5 below, present the two canines that were treated with the placebo for 150 days. Pain levels presented with no significant difference throughout the study as compared to the baseline values on Day 0.

TABLE 5
Effects of Placebo on pain from Limb
Manipulation in Arthritic Canines

| Day | Dog | Level | Mean ± SEM |
|---|---|---|---|
| 0 | J | 2.5 | 2.75 ± 0.12 |
| | K | 3 | |
| 30 | J | 2.5 | 2.6 ± 0.06 |
| | K | 2.75 | |
| 60 | J | 2.5 | 2.75 ± 0.12 |
| | K | 3 | |
| 90 | J | 3 | 3.0 ± 0.00 |
| | K | 3 | |
| 120 | J | 2.5 | 2.75 ± 0.12 |
| | K | 3 | |
| 150 | J | 2.5 | 2.75 ± 0.12 |
| | K | 3 | |

Pain after Physical Exertion.

Pain levels after physical exertion were evaluated in eleven canines over a period of 150 days with 30 days increments, including baseline values on Day 0. Levels of pain after physical exertion were observed on a scale of 0 to 4 with moderately arthritic canines presenting at levels at approximately 2-2.5. Observations made to determine pain levels included vocalization, lameness during or after exertion, body position, flexibility, integrity, and presence of crepitance.

As shown in Table 6 below, results for baseline values observed on Day 0 (1.81±0.18). This represents the 9 treated canines and they were observed to be mild to moderately arthritic with pain after exertion. Pain levels by Day 90 were significantly (P<0.05) decreased (0.83±0.19). Pain levels on Days 120 and 150 were further decreased (0.58±0.16; 0.42±0.14, respectively).

TABLE 6
Effects of Crominex ®3+ on Pain after
Physical Exertion in Arthritic Canines

| Day | Dog | Level | Mean ± SEM |
|---|---|---|---|
| 0 | A | 2.5 | 1.81 ± 0.18 |
| | B | 2 | |
| | C | 2.25 | |
| | D | 1 | |
| | E | 1 | |
| | F | 1.5 | |
| | G | 2 | |
| | H | 2 | |
| | I | 2 | |
| 30 | A | 2 | 1.53 ± 0.15 |
| | B | 1.5 | |
| | C | 2 | |
| | D | 1 | |
| | E | 1 | |
| | F | 1 | |
| | G | 1.75 | |
| | H | 2 | |
| | I | 1.5 | |
| 60 | A | 1.5 | 1.25 ± 0.13 |
| | B | 1 | |
| | C | 2 | |
| | D | 1 | |
| | E | 1 | |
| | F | 1 | |
| | G | 1 | |
| | H | 1.75 | |
| | I | 1 | |
| 90 | A | 1 | 0.83 ± 0.19* |
| | B | 1 | |
| | C | 1.5 | |
| | D | 0.5 | |
| | E | 0 | |
| | F | 0 | |
| | G | 1 | |
| | H | 1.5 | |
| | I | 1 | |
| 120 | A | 1 | 0.58 ± 0.16* |
| | B | 0.5 | |
| | C | 1 | |
| | D | 0 | |
| | E | 0 | |
| | F | 0 | |
| | G | 1 | |
| | H | 1 | |
| | I | 0.75 | |
| 150 | A | 0.75 | 0.42 ± 0.14* |
| | B | 0 | |
| | C | 0.75 | |
| | D | 0 | |
| | E | 0 | |
| | F | 0 | |
| | G | 0.5 | |
| | H | 1 | |
| | I | 0.75 | |

*Significantly different from the value of Day 0 (P < 0.05)

Results, as shown in Table 7 below, present the two canines that were treated with the placebo for the entire 150 days. Pain levels presented with no significant difference compared to baseline levels on Day 0.

TABLE 7
Effects of Placebo on Pain after physical
Exertion in Arthritic Canines

| Day | Dog | Level | Mean ± SEM |
|---|---|---|---|
| 0 | J | 1.5 | 1.75 ± 0.12 |
| | K | 2 | |
| 30 | J | 1.5 | 1.75 ± 0.12 |
| | K | 2 | |
| 60 | J | 2 | 2.0 ± 0.00 |
| 90 | J | 2 | 2.25 ± 0.12 |
| | K | 2.5 | |
| 120 | J | 2 | 2.0 ± 0.00 |
| | K | 2 | |
| 150 | J | 2.25 | 2.25 ± 0.00 |
| | K | 2.25 | |

Observation of Ortolani and Cranial Tibial Drawer Exam.

Along with the external evaluations of pain such as gait, or lameness, there are other options that may be performed during the physical exam. The Ortolani Maneuver is a common test performed on canines that are predisposed to hip dysplasia such as German Shepherds or larger breed canines. Ortiloni is performed on the hip joint by flexing the knee and hip to ninety degrees, placing the index finger on the greater trochanters, and abducting the hip (Ortolani's sign, 2007). As the hip is abducted, or moved away from the body, a positive Ortolani will present with a “clunk” sound or feeling as the femoral head relocate anteriorly to the acetabulum, or hip socket. As shown in Table 8, results present there was no significant or positive Ortolani sign in canines treated with Crominex®3+ from Day 0 to Day 150. The Ortolani is typically used on patients that have been sedated to receive a true positive or negative sign. Although the canines are moderately arthritic they may exhibit a negative Ortolani sign. As shown in Table 9, results present the two canines treated with the placebo. Both canines also did not exhibit a positive Ortolani sign from baseline values at Day 0 through Day 150.

TABLE 8
Ortolani's sign of Arthriticc canines treated with Crominex ®3+

| Day | Dog | Result | Day | Dog | Result |
|---|---|---|---|---|---|
| 0 | A | Negative | 90 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |
| 30 | A | Negative | 120 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |
| 60 | A | Negative | 150 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |

TABLE 9
Ortolani's sign of Arthritic Canines Treated with Placebo

| Day | Dog | Result | Day | Dog | Result |
|---|---|---|---|---|---|
| 0 | J | Negative | 90 | J | Negative |
| | K | Negative | | K | Negative |
| 30 | J | Negative | 120 | J | Negative |
| | K | Negative | | K | Negative |
| 60 | J | Negative | 150 | J | Negative |
| | K | Negative | | K | Negative |

Cranial Tibial Drawer is another test that can be performed upon physical exam to indicate arthritic changes and to diagnose the rupture of the cranial cruciate ligament (CCL). In this procedure the canine is in laterally recumbency with the veterinarian located behind the patient. The thumb of one hand is placed on the caudal aspect of the femoral condular region, and the index finger of the same hand is placed over the patella. The thumb of the other hand is placed on the head of the fibula, and the index finger is placed on the tibial crest (Devine, 1993). A positive tibial drawer is elicited with the ability to move the tibia cranially or forward in respect to the fixed femur. As shown in Table 10, results present with all canines treated with Crominex®3+ elicited a negative tibial drawer sign starting with baseline values on Day 0 through Day 150. As shown in Table 11, results present canines that were treated with the placebo also elicited a negative tibial drawer sign starting on Day 0 through Day 150.

TABLE 10
Cranial Tibial Drawer sign of Arthriticc canines
treated with Crominex ®3+

| Day | Dog | Result | Day | Dog | Result |
|---|---|---|---|---|---|
| 0 | A | Negative | 90 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |
| 30 | A | Negative | 120 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |
| 60 | A | Negative | 150 | A | Negative |
| | B | Negative | | B | Negative |
| | C | Negative | | C | Negative |
| | D | Negative | | D | Negative |
| | E | Negative | | E | Negative |
| | F | Negative | | F | Negative |
| | G | Negative | | G | Negative |
| | H | Negative | | H | Negative |
| | I | Negative | | I | Negative |

TABLE 11
Cranial Tibial Drawer sign of Arthritic Canines Treated with Placebo

| Day | Dog | Result | Day | Dog | Result |
|---|---|---|---|---|---|
| 0 | J | Negative | 90 | J | Negative |
| | K | Negative | | K | Negative |
| 30 | J | Negative | 120 | J | Negative |
| | K | Negative | | K | Negative |
| 60 | J | Negative | 150 | J | Negative |
| | K | Negative | | K | Negative |

Effects of Crominex®3+ on Body Weight, Heart Rate, Respiration Rate, Temperature and Biological Parameters in Serum.

Body weight, heart rate, respiration rate, temperature, and biological serum parameters were evaluated in eleven canines over a period of 150 days in increments of 30 days.

Table 12 below presents the results of body weight in kilograms for the nine Crominex®3+ treated canines. Body weight was recorded using the electronic scale at the Carman Pavilion. There were some fluctuations in body weight in the canines, with some exceeding the weight limitations. The body weight expressed in terms of Kg (mean±SEM) on Day 0 was 24.8±1.8; and on day 150 was 25.3±2.0. Table 13 below presents the data of body weight of two canines treated with the placebo. No significant changes were observed in either the treated group or placebo group for the entire 150 days (P>0.05).

TABLE 12
Effects of Crominex ®3+ on Body
Weight (Kg) of Arthritic Canines

| Day | Dog | Weight | Mean ± SEM |
|---|---|---|---|
| 0 | A | 19 | 24.8 ± 1.8 |
| | B | 20.3 | |
| | C | 20 | |
| | D | 29.5 | |
| | E | 30.1 | |
| | F | 31.8 | |
| | G | 22.1 | |
| | H | 19.5 | |
| | I | 30.5 | |
| 30 | A | 18.9 | 24.8 ± 1.8 |
| | B | 21.3 | |
| | C | 19.3 | |
| | D | 29.1 | |
| | E | 29.6 | |
| | F | 32.2 | |
| | G | 21.9 | |
| | H | 20.0 | |
| | I | 31.1 | |
| 60 | A | 19.2 | 24.8 ± 2.1 |
| | B | 19.2 | |
| | C | 18.9 | |
| | D | 30.4 | |
| | E | 29.3 | |
| | F | 33.4 | |
| | G | 21.2 | |
| | H | 20.1 | |
| | I | 31.9 | |
| 90 | A | 18.9 | 25.1 ± 2.0 |
| | B | 22.3 | |
| | C | 18.7 | |
| | D | 30.2 | |
| | E | 28.5 | |
| | F | 34.4 | |
| | G | 20.9 | |
| | H | 20.3 | |
| | I | 31.5 | |
| 120 | A | 19.5 | 25.2 ± 2.0 |
| | B | 22.6 | |
| | C | 18.6 | |
| | D | 31.0 | |
| | E | 27.9 | |
| | F | 34.8 | |
| | G | 21.1 | |
| | H | 20.1 | |
| | I | 31.1 | |
| 150 | A | 19.7 | 25.3 ± 2.0 |
| | B | 22.7 | |
| | C | 18.5 | |
| | D | 31.8 | |
| | E | 27.6 | |
| | F | 35.1 | |
| | G | 21.5 | |
| | H | 20.3 | |
| | I | 30.5 | |

No statistical significant difference from the value of Day 0 (P > 0.05)

TABLE 13
Effect on Weight (Kg)of Arthritic Canines Treated with Placebo

| Day | Dog | Level | Mean ± SEM |
|---|---|---|---|
| 0 | J | 26.7 | 27.1 ± 0.16 |
| | K | 27.4 | |
| 30 | J | 25.9 | 26.9 ± 0.45 |
| | K | 27.8 | |
| 60 | J | 25.1 | 26.4 ± 0.59 |
| | K | 27.6 | |
| 90 | J | 25.5 | 26.7 ± 0.54 |
| | K | 27.8 | |
| 120 | J | 24.7 | 26.6 ± 0.87 |
| | K | 28.4 | |
| 150 | J | 24.3 | 26.4 ± 0.99 |
| | K | 28.5 | |

Table 14 below presents the data of heart rate in beats per minute for the nine Crominex®3+ treated canines. Heart rate was observed and recorded using a stethoscope. Some canines presented with higher heart rates when arriving nervous, or excited, but levels remained within normal limits. The heart rate (mean±SEM) observed on Day 0 was 124.7±2.6, and on Day 150 was 125.6±3.0. Results in Table 15 below present the data of heart rate in two canines treated with the placebo. No significant changes were observed in either the treated or the placebo group for the entire 150 days (P>0.05).

TABLE 14
Heart Rate (Beats/Minute) of Arthritic Canines
Treated with Crominex ®3+

| Day | Dog | BPM | Mean ± SEM |
|---|---|---|---|
| 0 | A | 120 | 124.7 ± 2.6 |
| | B | 112 | |
| | C | 124 | |
| | D | 124 | |
| | E | 128 | |
| | F | 116 | |
| | G | 132 | |
| | H | 136 | |
| | I | 130 | |
| 30 | A | 124 | 124.7 ± 2.9 |
| | B | 112 | |
| | C | 120 | |
| | D | 116 | |
| | E | 132 | |
| | F | 120 | |
| | G | 130 | |
| | H | 140 | |
| | I | 128 | |
| 60 | A | 130 | 128.2 ± 2.5 |
| | B | 116 | |
| | C | 128 | |
| | D | 124 | |
| | E | 128 | |
| | F | 120 | |
| | G | 136 | |
| | H | 140 | |
| | I | 132 | |
| 90 | A | 128 | 124.9 ± 2.6 |
| | B | 108 | |
| | C | 132 | |
| | D | 120 | |
| | E | 128 | |
| | F | 120 | |
| | G | 132 | |
| | H | 132 | |
| | I | 124 | |
| 120 | A | 116 | 126.7 ± 3.5 |
| | B | 112 | |
| | C | 128 | |
| | D | 128 | |
| | E | 132 | |
| | F | 116 | |
| | G | 136 | |
| | H | 144 | |
| | I | 128 | |
| 150 | A | 120 | 125.6 ± 3.0 |
| | B | 112 | |
| | C | 116 | |
| | D | 128 | |
| | E | 124 | |
| | F | 124 | |
| | G | 136 | |
| | H | 140 | |
| | I | 130 | |

No statistical significant difference from the value of Day 0 (P > 0.05)

TABLE 15
Heart Rate (Beats/Minute) of Arthritic
Canines Treated with Placebo

| Day | Dog | Level | Mean ± SEM |
|---|---|---|---|
| 0 | J | 112 | 112 ± 0.00 |
| | K | 112 | |
| 30 | J | 112 | 124 ± 5.7 |
| | K | 136 | |
| 60 | J | 132 | 122 ± 4.7 |
| | K | 112 | |
| 90 | J | 140 | 138 ± 0.9 |
| | K | 136 | |
| 120 | J | 128 | 134 ± 2.8 |
| | K | 140 | |
| 150 | J | 136 | 140 ± 1.9 |
| | K | 144 | |

Table 16 data below presents respiration rate in breaths per minute for the nine treated canines Respiration rate was recorded using a stethoscope. Some canines were panting and levels could not be recorded, and therefore only levels on Day 120 could be measured for the average. Since values could not be measured, the data could not be analyzed statistically. Table 17 below presents the data of respiration rate in two canines that were treated with the placebo.

TABLE 16
Respiration (Beats/Minute) of Arthritic Canines
Treated with Crominex ®3+

| Day | Dog | RR/Min | Mean ± SEM |
|---|---|---|---|
| 0 | A | 32 | N/A |
| | B | Pant | |
| | C | 28 | |
| | D | Pant | |
| | E | 20 | |
| | F | 32 | |
| | G | Pant | |
| | H | 32 | |
| | I | 28 | |
| 30 | A | 28 | N/A |
| | B | Pant | |
| | C | 32 | |
| | D | 40 | |
| | E | Pant | |
| | F | 28 | |
| | G | 32 | |
| | H | 36 | |
| | I | 32 | |
| 60 | A | 28 | N/A |
| | B | Pant | |
| | C | 20 | |
| | D | 28 | |
| | E | Pant | |
| | F | 28 | |
| | G | Pant | |
| | H | 32 | |
| | I | 32 | |
| 90 | A | 28 | N/A |
| | B | Pant | |
| | C | 44 | |
| | D | 32 | |
| | E | 28 | |
| | F | 20 | |
| | G | 28 | |
| | H | Pant | |
| | I | pant | |
| 120 | A | 32 | 33.3 ± 2.58 |
| | B | 48 | |
| | C | 40 | |
| | D | 28 | |
| | E | 20 | |
| | F | 32 | |
| | G | 32 | |
| | H | 32 | |
| | I | 36 | |
| 150 | A | 32 | N/A |
| | B | 32 | |
| | C | 32 | |
| | D | 28 | |
| | E | 32 | |
| | F | 32 | |
| | G | 36 | |
| | H | 28 | |
| | I | Pant | |

No statistical significant difference from the value of Day 0 (P > 0.05)

TABLE 17
Respiration (Beats/Minute) of Arthritic
Canines Treated with Placebo

| Day | Dog | Level | Mean ± SEM |
|---|---|---|---|
| 0 | J | 28 | 28 ± 0.00 |
| | K | 28 | |
| 30 | J | 36 | 32 ± 1.9 |
| | K | 28 | |
| 60 | J | 28 | 28 ± 0.00 |
| | K | 28 | |
| 90 | J | 28 | 32 ± 1.9 |
| | K | 36 | |
| 120 | J | 32 | N/A |
| | K | Pant | |
| 150 | J | 28 | 30 ± 0.9 |
| | K | 32 | |

Table 18 below presents the data of body temperature in ° F. for the nine-treated canines Temperature was recorded using a rectal digital thermometer. Levels did not change significantly (P>0.05) from Day 0 (102.1±0.35) to Day 150 (101.7±0.22). Table 19 below shows the data of body temperature in two canines treated with the placebo. No significant changes were observed in either the treated or placebo groups for the entire 150 days (P>0.05).

TABLE 18
Temperature (° F.) of Arthritic Canines Treated
with Crominex ®3+

| Day | Dog | Temp ° F. | Mean ± SEM |
|---|---|---|---|
| 0 | A | 100.4 | 102.1 ± 0.35 |
| | B | 102.8 | |
| | C | 103.3 | |
| | D | 102.5 | |
| | E | 101.2 | |
| | F | 103.1 | |
| | G | 102.0 | |
| | H | 101.0 | |
| | I | 103.0 | |
| 30 | A | 101.2 | 101.8 ± 0.25 |
| | B | 102.7 | |
| | C | 101.0 | |
| | D | 102.0 | |
| | E | 101.7 | |
| | F | 102.5 | |
| | G | 101.4 | |
| | H | 100.8 | |
| | I | 102.8 | |
| 60 | A | 100.4 | 101.7 ± 0.25 |
| | B | 102.5 | |
| | C | 101.9 | |
| | D | 101.5 | |
| | E | 101.5 | |
| | F | 103 | |
| | G | 101.3 | |
| | H | 101.4 | |
| | I | 101.6 | |
| 90 | A | 101.1 | 101.7 ± 0.15 |
| | B | 101.8 | |
| | C | 101.4 | |
| | D | 101.6 | |
| | E | 101.6 | |
| | F | 102.3 | |
| | G | 101.5 | |
| | H | 101.5 | |
| | I | 102.5 | |
| 120 | A | 100.8 | 101.7 ± 0.16 |
| | B | 102.1 | |
| | C | 101.5 | |
| | D | 101.9 | |
| | E | 101.7 | |
| | F | 102.2 | |
| | G | 101.8 | |
| | H | 101.4 | |
| | I | 102.3 | |
| 150 | A | 100.7 | 101.7 ± 0.22 |
| | B | 102.3 | |
| | C | 101.1 | |
| | D | 102.0 | |
| | E | 101.2 | |
| | F | 102.5 | |
| | G | 101.3 | |
| | H | 101.4 | |
| | I | 102.4 | |

No statistical significant difference from the value of Day 0 (P > 0.05)

TABLE 19
Temperature (° F.) of Arthritic Canines Treated with Placebo

| Day | Dog | Level | Mean ± SEM |
|---|---|---|---|
| 0 | J | 102.0 | 101.7 ± 0.14 |
| | K | 101.4 | |
| 30 | J | 101.8 | 101.3 ± 0.24 |
| | K | 100.8 | |
| 60 | J | 101.6 | 102.0 ± 0.16 |
| | K | 102.3 | |
| 90 | J | 102.0 | 101.9 ± 0.05 |
| | K | 101.8 | |
| 120 | J | 102.2 | 102.6 ± 0.19 |
| | K | 103.0 | |
| 150 | J | 102.7 | 102.6 ± 0.07 |
| | K | 102.4 | |

Table 20 below presents the data of serum chemistry parameters from the nine treated canines Parameters included BUN, creatinine, total bilirubin, ALT, creatine kinase, and AST. Blood samples were collected from the jugular vein and the serum collected was sent to the Breathitt Veterinary Center for analysis. In one of the treated canines, ALT levels increased for a one month period, but other parameters remained within normal range. All other levels for the nine canines remained within normal range throughout the entire 150 days (P>0.05). Table 21 below presents the serum chemistry data in two canines treated with the placebo and levels remained statistically unchanged throughout the entire 150 days (P>0.05).

TABLE 20
Serum Chemistry Parameters of Canines Treated with Crominex ®3+

| Parameter | Unit | DAY 0 | DAY 30 | DAY 60 | DAY 90 | DAY 120 | DAY 150 |
|---|---|---|---|---|---|---|---|
| BUN | mg/dL | 15.1 ± 1.21 | 14.7 ± 1.2 | 15.8 ± 1.35 | 16.1 ± 1.34 | 14.4 ± 1.07 | 14.8 ± 1.26 |
| Creatinine | mg/dL | 0.89 ± .09 | 0.90 ± .10 | 0.91 ± .10 | 0.84 ± .08 | 0.90 ± .11 | 0.86 ± .08 |
| T Bilirubin | mg/dL | 0.20 ± .03 | 0.19 ± .01 | 0.22 ± .04 | 0.18 ± .01 | 0.17 ± .04 | 0.19 ± .01 |
| ALT | IU/L | 60.9 ± 15.1 | 102.2 ± 47.8 | 59.2 ± 13.3 | 66.0 ± 14.8 | 62.0 ± 15.3 | 57.7 ± 14.3 |
| AST | IU/L | 26.3 ± 1.9 | 24.1 ± 1.4 | 27.9 ± 3.2 | 23.8 ± 1.81 | 24.2 ± 2.11 | 24.6 ± 2.3 |
| CK | IU/L | 160.4 ± 24.6 | 124.4 ± 29.7 | 231.3 ± 99.5 | 108.2 ± 16.02 | 130.2 ± 45.3 | 150 ± 31.6 |

No statistically significant difference from the value of Day 0 (P > 0.05)

TABLE 21
Serum Chemistry Parameters of Canines Treated with Placebo

| Parameter | Unit | DAY 0 | DAY 30 | DAY 60 | DAY 90 | DAY 120 | DAY 150 |
|---|---|---|---|---|---|---|---|
| BUN | mg/dL | 13.5 ± 0.24 | 9.5 ± 0.24 | 9.5 ± 0.71 | 12.0 ± 0.5 | 12.0 ± 0.00 | 10.0 ± 0.00 |
| Creatinine | mg/dL | 0.94 ± 0.09 | 0.87 ± 0.03 | 0.91 ± 0.05 | 0.92 ± 0.1 | 0.97 ± 0.04 | 0.83 ± .07 |
| T Bilirubin | mg/dL | 0.15 ± 0.02 | 0.2 ± 0.02 | 0.2 ± 0.00 | 0.2 ± 0.00 | 0.2 ± 0.00 | 0.2 ± 0.00 |
| ALT | IU/L | 45.5 ± 13.0 | 122 ± 24.7 | 114 ± 22.6 | 57.0 ± 6.84 | 66.0 ± 10.8 | 52.5 ± 16.3 |
| AST | IU/L | 23.0 ± 2.4 | 14.0 ± 2.6 | 23.0 ± 0.2 | 17.0 ± 2.83 | 15.0 ± 2.1 | 24.0 ± 2.8 |
| CK | IU/L | 208 ± 44.3 | 68 ± 11.5 | 153 ± 16.5 | 85.0 ± 1.65 | 105.0 ± 1.2 | 196.0 ± 52.8 |

No statistically significant difference from the value of Day 0 (P > 0.05)

Moderately arthritic dogs treated with Crominex®3+ daily (25 mg twice) for a period of 150 days significantly ameliorated arthritic pain. Not only did the owners report that their pets increased their activity, but they also reported that their pets were able to jump on the bed and climb steep stairways. One owner reported her pet performed her “happy dance” (i.e., behavior demonstrating playful excitement and high spirits, which can include leaping, twisting and/or spinning about, or the like) which she had not performed in years, and another started to play with a ball which he had not done for years. The FIGURES discussed in the next section provide graphical evidence of the significant decrease in pain levels over the course of 150 days.

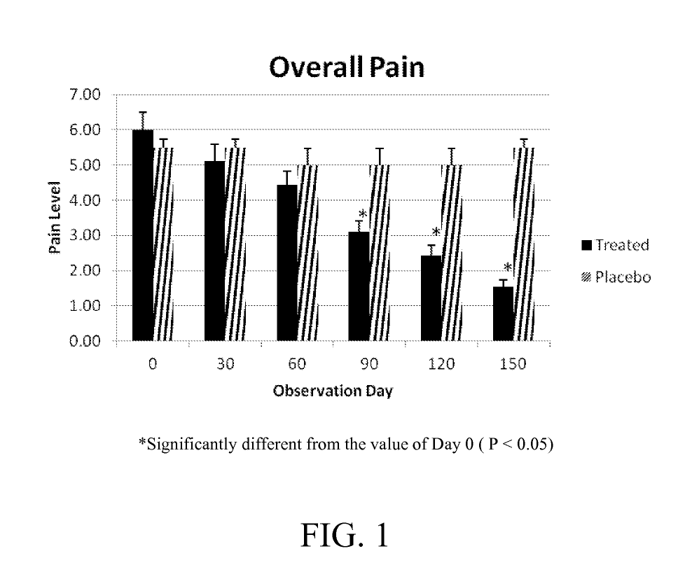

FIG. 1 presents with the overall pain levels for the treated and placebo groups with a significant difference reported beginning on Day 90. Levels of pain began to further decrease on Days 120 and 150 with two of the canines receiving the most benefits from administration of Crominex®3+. All nine of the treated canines benefitted from the treatment and pain levels decreased from moderate to very mild over the entire course of the 150 days.

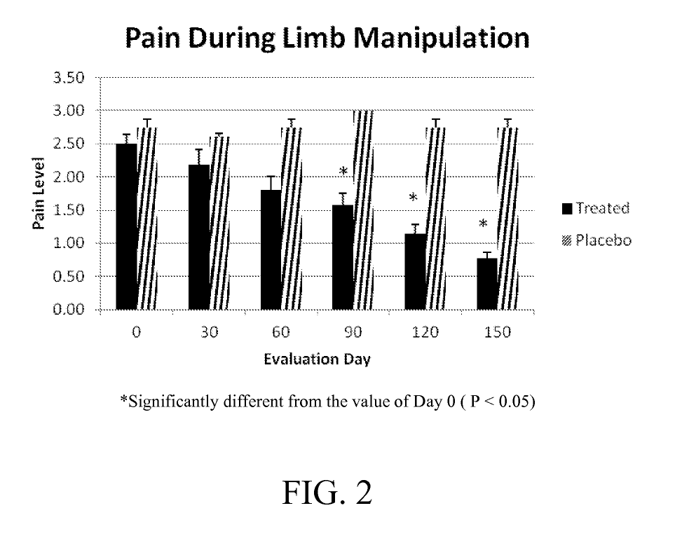

FIG. 2 presents the pain levels on manipulation of the limbs for the treated and placebo groups. A significant difference was noted beginning on Day 90 in Crominex®3+ treated dogs. Levels continued to decrease on Days 120 and 150 with several canines receiving the most benefit from administration of Crominex®3+. All nine of the treated canines benefitted from the treatment, and pain levels decreased from moderate to very mild over the entire course of the 150 days.

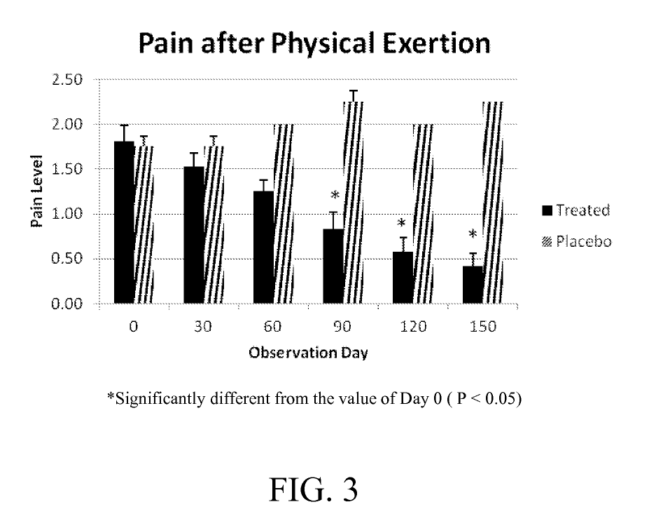

FIG. 3 presents the pain levels after physical exertion for the treated and placebo groups. A significant difference was noted beginning on Day 90 in Crominex®3+ treated dogs. Levels continued to decrease on Days 120 and 150 with all canines presenting significant benefit from the administration of Crominex®3+. Levels of pain on Day 0 were not very high, but pain levels decreased from semi-moderate to very mild or non-existent over the entire 150 days. Owners also observed their canines were observed with more willingness to go for walks, and run while playing.

Lateral radiographs of the elbow, carpus, tarsus, hock, and a ventral dorsal view of the hip joints were observed to visualize osteophyte reduction over the 150 days. The images were performed on Day 0 and 150 to achieve a comparison. The quality of the ventral dorsal hip joints created a challenge to visualize any osteoarthritic changes. This is an uncomfortable position for a canine and movement can cause issues during a radiograph. In the lateral limb views, some images on Day 0 varied from the quality of views on Day 150. Problems could have occurred as the same technique may not have been used for each limb on Day 0 and Day 150.

Physical examinations were performed on every canine throughout the entire 150 days to ensure the safety of Crominex®3+. Blood serum samples were sent to Breathitt Veterinary Center to evaluate kidney, liver, and heart muscle functions. No significant changes were observed as only one canine had a slight increase in ALT for one month during the study. Body weight, heart rate, respiration rate, and temperature were also evaluated each month for any significant changes. Body weight did fluctuate slightly in some canines, but may been affected by diet changes, or a decrease in exercise during the winter months. Heart rate remained steady in each canine and some increases were due to excitement. Respiration rates also fluctuated slightly as excitement accounted for some of the canines panting. There were no owner complaints on inappetence, vomiting, or any other side effects. Crominex®3+ was well tolerated by the canines, and has been proven safe for moderately arthritic canines.

In conclusion, the results presented from overall pain, pain during limb manipulation, and pain after physical exertion conclude that administering Crominex®3+ (25 mg) twice daily provides significant relief from pain and inflammation associated with canine arthritis. Physical examination and blood serum analysis also addressed no side effects in canines treated with Crominex®3+. The two canine that receive the placebo exhibited no significant change or improvement during the entire duration of 150 days. Although the placebo did not show a change in pain levels, no side effects of organ function were observed. Through physical examination, pain level analysis, and serum chemistry analysis, Crominex®3+ has been proven effective, and presents as a safe alternative to treat canine arthritis.

It is further expected that when Crominex®3+ is administered to human patients, similar beneficial results will be observed. It is further expected that Crominex®3+ will be effective upon administration to other mammals or domestic animals.

The methods described above may be further understood in connection with the following Examples. The present study (Examples B, C, and D) was a prospective, randomized, double blinded, double dummy, and placebo controlled trial conducted in the Department of Clinical Pharmacology and Therapeutics, Nizam's Institute of Medical Sciences, Hyderabad, India. A total of hundred patients were screened and 90 patients were enrolled to receive the study treatment (and of these a subset was selected for treatment in the present examples) in a randomized manner. The study was approved by the Institutional Ethics Committee and all the subjects gave written informed consent prior to their participation in the study.

### Example A

It is further expected that treatment of a human population with Crominex®3+ as exemplified above would reduce overall pain, and/or pain on limb movement, manipulation, or other exertion in a statistically significant manner. For example, one or more human individuals are administered an effective daily dose of between about 20-50 mg Crominex®3+ to achieve reduction of one or more of the listed symptoms of arthritis. The daily dose of Crominex®3+ is administered for a period of time at least until symptoms are decreased. In an embodiment, and effective dose is between about 10-25 mg Crominex®3+ administered twice daily to achieve reduction of one or more of the listed symptoms of arthritis.

It was observed in human clinical trials that a daily dose of about 20-24 mg Crominex®3+ (equivalent to a 400 mcg dose of Cr 3+) administered orally was effective against a number of osteoarthritis outcome measures, as shown in the following Examples.

### Example B

Clinical Study. A randomized, double-blind, placebo-controlled, parallel-group study to evaluate the safety and analgesic effect of Crominex in subjects with Osteoarthritis.

Osteoarthritis patients of either gender aged between 40 and 70 years for at least 6 months duration and meeting the ARA functional class I to III and radiological evidence of osteoarthritis. Only patients who have grade II to IV of the Kellgren and Lawrence scale in the knee joint X-ray and who record baseline pain scores of at least 40 mm on the VAS (visual analogue scale) monitored at baseline visit were enrolled. Patients who were willing to discontinue all current analgesic therapy, including NSAIDs, OTC pain medications and topical analgesics were enrolled into the study. Patients with severe osteoarthritis (ARA functional class IV) were excluded from the study. Patients with radiological grading—Kellgren and Lawrence scale ranging from grade 0 to grade I, patients on alternative system of medicine, any psychiatric disorder or who have been using systemic/Intra-articular steroids within 12 weeks and hyaluronic acid in the last 9 months, or potential candidates for imminent joint replacement were also excluded. Patients with uncontrolled hypertension or diabetes, hepatic or renal impairment, pregnant or lactating females, or with a recent trauma of the involved knee were excluded from the study.

After screening, a subset of all the eligible patients were randomized to either of two (2) treatment groups in a double blinded and double dummy fashion for duration of 12 weeks: 1. Crominex Group (n=20)—1 capsule of 400 mcg orally once a day and one capsule of an identical placebo twice daily after food. 2. Identical Placebo Group (n=20)—two capsules in the morning and one in the evening after food.

Subjects were asked to review for follow-up visits at 4 weeks, 8 and 12 weeks of therapy. At each visit they were evaluated for efficacy and safety. The patients were assessed using Modified WOMAC index scale (mWOMAC), Knee swelling index and Visual analogue scale (VAS) for pain, stiffness and disability, all of which were recorded at baseline and end of study treatment (12 weeks). The patients were allowed to take paracetamol tablets as rescue medication and the total count of rescue medication used was recorded at the end of the study. Safety lab investigations for hematological, hepatic and renal biochemical parameters were conducted before and at the end of the study and also as and when required (in case of any adverse drug reaction (ADR)). Subjects were enquired for the presence of ADR and the same was recorded in the case report form. Compliance to therapy was assessed by pill count method.

Study Procedure.

Patients were enrolled in the present study after reading, understanding and signing the informed consent form. Then they were screened and assessed for the inclusion/exclusion criteria (visit 1). At the baseline/randomization visit (visit 2, day 1), vital signs, general examination, routine lab investigations, modified WOMAC scoring, VAS for subjective assessment of pain, stiffness and disability respectively, swelling index for the involved knee joint were performed and all eligible subjects were randomized into the study medication to receive either one of the five treatments as per prior randomization schedule. The study medication and rescue medication (Paracetamol 650 mg) were dispensed at every visit and compliance checked by pill count method at every visit. Rescue medication accountability is performed to find out rescue medication consumption during the treatment period.

The subsequent 3 visits were scheduled at 4 weeks intervals (visit 3 occurred after 4 weeks of treatment, visit 4 after 8 weeks of treatment), vital signs, General examination, modified WOMAC scoring, VAS for subjective assessment of pain, stiffness and disability respectively, swelling index for the involved knee joint, pill count for study and rescue medication were performed and each patient received another supply of the trial medication and rescue medication. At the conclusion of the study, at Visit 5 (after 12 weeks post treatment), vital signs, General examination, routine safety lab investigations, examination of the affected knee by modified WOMAC scoring, VAS for subjective assessment of pain, stiffness and disability respectively, and swelling index for the involved joint were performed. At every visit each patient was assessed regarding any incidence of adverse effect especially GI intolerance and same noted in case record form. Adverse Effects/SAE monitoring was performed throughout the course of the study. Safety lab parameters will be done before and after treatment and as and when required. Each participant is given a contact number for reporting and accessing medical help with regard to any adverse event.

Primary Outcome Measures (of Example B).

Modified Western Ontario and McMaster University OA Index (mWOMAC) is a disease specific outcome measure for osteoarthritis. It has three subscales assessing pain—A (5 questions), stiffness—B (2 questions) and physical function for disability—C (17 questions). This outcome was measured at baseline, week 4, week 8 and week 12. In this study the primary outcome was the reduction in modified WOMAC total score (A+B+C) from baseline to the end of treatment at week 12.

Secondary Outcome Measures.

1. VAS based assessment of Pain, Disability, and Stiffness subscales (i.e. subjective assessment of the mWOMAC subscales). Pain Subscale is assessed by: no pain (0 mm) to extreme pain (100 mm)—see, Example D. Stiffness Subscale is assessed by: no stiffness (0 mm) to extreme stiffness (100 mm)—see, Example D. Disability (i.e., Physical Function) Subscale is assessed by: no disability (0 mm) to extreme disability (100 mm)—see, Example D.

2. Swelling index (KSI) as measured by signal joint knee circumference (in mm)—see, Example C.

3. Use of rescue medication, i.e., 650 mg paracetamol, in all treatment groups.

4. Physician global assessment, characterized by 5 categories: Excellent—complete relief of symptoms; Good—partial relief of symptoms; Fair—minimal relief of symptoms; Poor—no relief of symptoms; Very Poor—worsening of symptoms.

Statistical Analysis.

Data are expressed as mean±SD. Primary and secondary outcome measures were analyzed as the absolute change and mean percentage reduction in the response over the 12-week treatment period. Paired ‘t’ test was used to compare the mean change from baseline to post treatment within group and unpaired “t” test for between group comparisons. All statistical analysis was performed using the Graph pad PRISM software 4 (Graph pad software Inc. San Diego, Calif., USA).

Results.

A total of 40 eligible patients have completed the study, that is, 20 patients each in Crominex and placebo groups have completed the study.

TABLE 22
DEMOGRAPHIC DATA

| | Crominex | Placebo |
|---|---|---|
| | Group | Group |
| Total No. | 20 | 20 |
| Gender (M/F) | 13/7 | 12/8 |
| Age (yrs) | 55.9 ± 9.06 | 58.05 ± 6.07 |
| Weight (Kg) | 61.63 ± 6.43 | 75.9 ± 8.24 |
| BMI (Kg/m²) | 26.14 ± 2.03 | 28.5 ± 3.73 |

The detailed demographic characteristics of the study groups are shown in Table 22. There were no significant differences between treatment groups in baseline characteristics including age, weigh, and body mass index.

TABLE 23
Modified WOMAC score

| | Crominex | Placebo |
|---|---|---|
| | Group | Group |
| BASELINE | 53.5 ± 5.89 | 51.95 ± 6.53 |
| END OF 12 | 44.7 ± 2.63* | 49.5 ± 6.13* |
| WEEKS | | |
| ABSOLUTE | 8.8 ± 4.61* | 2.45 ± 3.07* |
| CHANGE | | |

*P value <0.001, compared to baseline in both groups, and between groups (absolute change)

As shown in Table 23, the baseline values of modified WOMAC score were comparable in both treatment groups. There was significant reduction in the modified WOMAC score after 12 weeks of treatment compared to baseline in both treatment groups (P value <0.001).

When the absolute change in reduction of modified WOMAC scores was compared between treatment groups, it was found to be extremely significant with a P value <0.001.

When the mean percentage reduction of modified WOMAC scores was compared between treatment groups, it was found to be extremely significant with a P value <0.001. Specifically, the mean percent reduction was found to be 16.4% in the Crominex group vs. 4.7% in the Placebo group.

### Example C

TABLE 24
KNEE SWELLING INDEX (KSI)

| | Crominex | Placebo |
|---|---|---|
| | Group | Group |
| BASELINE | 364.8 ± 21.30 | 404.1 ± 25.79 |
| END OF 12 | 349.1 ± 20.88* | 393.8 ± 25.45* |
| WEEKS | | |
| ABSOLUTE | 15.7 ± 7.59*@ | 10.3 ± 3.8*@ |
| CHANGE | | |

*P value <0.001, compared to baseline in both groups
@P value <0.05 between groups

As shown in Table 24, the baseline values of knee swelling index were comparable in both treatment groups. There was significant reduction in knee swelling index after 12 weeks of treatment compared to baseline in both treatment groups (P value <0.001).

When the absolute change in reduction of Knee Swelling Index was compared between treatment groups, it was found to be significant with a P value <0.05 as shown in Table 24.

When the mean percentage reduction of Knee Swelling Index was compared between treatment groups, it was found to be highly significant with a P value <0.01. Specifically, the mean percent reduction was found to be 4.3% in the Crominex group vs. 2.5% in the Placebo group.

### Example D

For the VAS-based measurements taken of VAS-Pain, VAS-Stiffness, and VAS-Disability, none demonstrated significant results when the Crominex group was compared to the Placebo group. That is, for each of the three outcome measures: absolute change was not found to be significant, and mean percent change was not found to be significant.

Rescue Medications.

The number of rescue medications that were used by the patients in both groups during the course of the study is shown in Table 25. It was found that the usage of Paracetamol 650 mg was much higher in the Placebo group (28±13.01).

TABLE 25
Use of Rescue medication (Paracetamol 650 mg)

| | Crominex | Placebo |
|---|---|---|
| | Group | Group |
| MEAN | 15 | 28 |
| SD | 1.34 | 13.01 |

Safety Assessments.

All safety haematological, hepatic and renal biochemical parameters were within normal limits with all treatment groups. In the Crominex 400 mcg group two subjects had diarrhoea. None of the patients had any serious side effect and no subjects discontinued the study due to adverse events.

It may be concluded from the present study that all the groups were homogenous in baseline characteristics. Treatment with Crominex 400 mcg for a period of 12 weeks in osteoarthritis patients demonstrated a significant reduction in modified WOMAC score and Knee swelling index when compared to baseline and placebo. Chromium alone is not known to have anti-inflammatory effects; however, it was unexpectedly shown that a chromium-containing composition comprising chromium 3+, an extract of Phyllanthus emblica, and Shilajit, unexpectedly reduced inflammation and specifically, symptoms of osteoarthritis.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof. Similarly, the compositions as described may be used for pharmaceutical compositions, cosmetic compositions, or skin care compositions, and may be administered in combination with a pharmaceutically or cosmeceutically acceptable carrier, as appropriate.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

Solid nutritional compositions for oral administration may optionally contain, in addition to the above enumerated nutritional composition ingredients or compounds: carrier materials such as, but not limited to, corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearates, stearic acid, silicone fluid, talc, waxes, oils, colloidal silica, and the like. The usefulness of such excipients is well known in the art.

In one embodiment, the nutritional composition may be in the form of a liquid. In accordance with this embodiment, a method of making a liquid composition is provided.

Liquid nutritional compositions for oral administration in connection with a method for preventing and/or treating arthritis, or inflammatory symptoms thereof, can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, a chromium-containing composition, or Phyllanthus emblica extract and/or Shilajit may be further combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearate, stearic acid, silicone fluid, talc, waxes, oils, colloidal silica, and the like. The usefulness of such excipients is well known in the art.

In one preferred embodiment, the nutritional composition may be in the form of a liquid. In accordance with this embodiment, a method of making a liquid composition is provided.

Liquid nutritional compositions for oral administration in connection with a method for preventing and/or treating inflammation, colds and/or flu can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

Routes of Administration

The compositions may be administered by any suitable route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped artices, e.g. films or microcapsules.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method of treating osteoarthritis in a mammal, comprising administering to the mammal in need thereof a therapeutically effective amount of a chromium-containing composition comprising chromium 3+, an extract of Phyllanthus emblica, Shilajit, and an acceptable carrier.

2. The method of claim 1, wherein the chromium-containing composition includes from about 400 mcg chromium 3+ to about 1000 mcg chromium 3+ per day.

3. The method of claim 2, wherein the chromium-containing composition is administered in a daily dosage from about 20 mg to about 50 mg per day, which is equivalent to about 400 mcg to about 1000 mcg of chromium 3+ per day.

4. The method of claim 1, wherein the mammal is selected from the group consisting of human, dog, cat, cow, horse, monkey, pig, sheep, cow, sheep, goat, and rabbit.

5. The method of claim 2 wherein the mammal is a human.

6. The method of claim 3 wherein the mammal is a human.

7. The method of claim 3 wherein the chromium-containing composition is administered orally.

8. A method of reducing pain and inflammation in an individual afflicted with osteoarthritis, comprising orally administering to the individual in need thereof a therapeutically effective amount of a chromium-containing composition comprising chromium 3+, an extract of Phyllanthus emblica, Shilajit, and an acceptable carrier.

9. The method of claim 8, wherein the chromium-containing composition includes from about 400 mcg chromium 3+ to about 1000 mcg chromium 3+ per day.

10. The method of claim 9, wherein the chromium-containing composition is administered in a daily dosage from about 20 mg to about 50 mg per day, which is equivalent to about 400 mcg to about 1000 mcg of chromium 3+ per day.

11. The method of claim 8, wherein the individual is treated for about 2 weeks to about 12 weeks.

12. The method of claim 11, further comprising measuring pain and inflammation by a mWOMAC index score, and wherein the mWOMAC score is reduced in a range from about 15% to about 20%.

13. A method of reducing pain and inflammation in a dog, comprising orally administering to the dog in need of such treatment a therapeutically effective amount of a chromium-containing composition comprising chromium 3+, an extract of Phyllanthus emblica, Shilajit, and an acceptable carrier.

14. The method of claim 13, wherein the chromium-containing composition includes from about 500 mcg chromium 3+ to about 1000 mcg chromium 3+ per day.

15. The method of claim 14, wherein the chromium-containing composition is administered in a daily dosage from about 25 mg to about 50 mg per day, which is equivalent to about 500 mcg to about 1000 mcg of chromium 3+ per day.

16. The method of claim 15, wherein the dog is treated for about 4 weeks to about 20 weeks.

17. The method of claim 16, further comprising measuring pain by limb manipulation or observation of physical exertion, and wherein pain observed during manipulation or physical exertion is reduced by at least about 50%.
